(19) United States
(12) Patent Application Publication
(10) Pub. No.: US 2015/0010594 A1
(43) Pub. Date: Jan. 8, 2015

(54) HIV VACCINE

(71) Applicant: Case Western Reserve University, Cleveland, OH (US)

(72) Inventor: Yong Gao, Cleveland, OH (US)

(21) Appl. No.: 14/322,545

(22) Filed: Jul. 2, 2014

Related U.S. Application Data
(60) Provisional application No. 61/841,972, filed on Jul. 2, 2013.

(51) Int. Cl. A61K 39/21; C12N 7/00

(52) U.S. Cl. CPC A61K 39/21; C12N 7/00; C12N 2740/16034; C12N 2740/16134; C12N 2740/16234
USPC 424/208.1

(57) ABSTRACT

A method for inducing an immune response against HIV in a subject includes preparing first and second HIV-1 protein coding sequences, introducing the first and second HIV-1 protein coding sequence into first and second expression constructs using yeast homologous recombination, transfecting a cell with the first and second, wherein the HIV-1 particle is secreted by the cell, and administering the secreted HIV-1 particle and a pharmaceutically acceptable carrier to the subject, wherein the secreted HIV-1 particle stimulates an immune response.

### GOVERNMENT FUNDING

This invention was made with government support under Grant No. AI49170 awarded by The National Institutes of Health, American Foundation of AIDS Research. The United States government has certain rights to the invention.

### BACKGROUND

The Human Immunodeficiency Virus (HIV) is the causative agent of Acquired Immunodeficiency Syndrome (AIDS). HIV rapidly undergoes genetic changes to escape from the subject's immune system response. Identification of potent, broadly cross-reactive human monoclonal antibodies to HIV has major implications for development of HIV inhibitors, vaccines, and tools for understanding mechanisms of HIV entry.

Eliciting and boosting immune responses by therapeutic vaccination has been used in HIV-1 patients. However, studies are limited, sample sizes are relatively small, and design of therapeutic vaccines is yet to be improved.

### SUMMARY

Embodiments described herein relate to an HIV based vaccine system that includes an HIV multivalent vector with diverse HIV envelope genes that are produced through forced recombination of the env region using of yeast-based cloning methods. The system can rapidly produce HIV envelope clones. The produced viruses are similar to wild-type HIV virus, and can mimic the entry process, expose hidden epitopes, complete reverse transcription, integrate into host chromosome, and continuously produce HIV envelope proteins, to elicit both humoral and cellular immune responses. In some embodiments, the produced env recombinants only contain break points in the gp41 region. In other embodiments, the vaccine system can be used to generate and/or screen for broadly neutralizing anti-HIV antibodies.

In some embodiments, the multivalent vaccines can be controlled to contain as few as 10 to greater than 1,000 unique variants where the recombination breakpoints in a quarter of the variant occurs within a codon to generate a nonsynonymous but functional amino acid substitution. The degree of functional heterogeneity of this heterologous subtype vaccine can be designed to be greater than in the autologous, heterogeneous HIV-1 vaccine. This diversity of the heterologous vaccine can be sufficient as a therapeutic vaccine.

### BRIEF DESCRIPTION OF THE DRAWINGS

FIGS. 1a and 1b illustrate a strategy for first examining the HIV-1 diversity in plasma and memory T cells form HIV-1 patients and representation of these populations in the autologous vaccine vectors and then determining the activation of memory T cells by autologous dendritic cells primed by different autologous vaccine candidates.

FIG. 2 illustrates cloning of HIV-1 genes/coding sequences derived from an HIV-1 patient into pREC_nfl_HIV-1/URA3 vector. (A) For creation of env chimera, the env gene is RT-PCR amplified from the patient sample and then transformed into yeast along with the linearized pREC_nfl_HIV-1Δenv/URA3 vector to obtain pREC_nfl_HIV-1/envpatient. (B) Colony growth is monitored on plates following selection with specific media. (C) The HIV-1env gene derived from three infected patients were inserted into pREC_nfl_HIV-1 Δenv/URA3 by yeast recombination and growth on C-Leu/5-FOA plates.

FIG. 3 is a schematic for the production and testing of an autologous multivalent vaccine.

FIG. 4 illustrates HIV env recombination system. (A, B) Schematic on the mechanism how the system produce pure and functional HIV intersubtype env recombinants; (C) Examples of successful production of virus from the system; (D) The produced env recombinants only contain breakpoints in gp41 region.

FIG. 5 illustrates a function analysis of HIV-1 envA/D recombinants. (A) Distribution of recombination breakpoints in env recombinants from dual infection with A91/D109; (B); In dual infection system, a few of env recombinants with breakpoints in gp41 but not in gp120 are functional; (C) All of the env recombinants from HIV-1 env recombination system contain breakpoints in gp41 and most of them are functional (D,E). 50% of gp120 recombinants converted to be functional after introduction of autologous C1/C5 region of gp120.

FIG. 6 illustrates immune response in huCD4 B cell transgenic mice vaccinated with multivalent anti-HIV vaccines. Group 4 received single envA/D recombinants-, and Group 5 and Group 6 received 25 different env recombinants or primary envs-based anti-HIV vaccines through sequential vaccination strategy.

FIG. 7 illustrates immune response in macaques inoculated with single SHIVenvB3, pool of SHIVenvB or SHIVenvC viruses.

### DETAILED DESCRIPTION

It should be understood that the present invention is not limited to particular methods, reagents, compounds, compositions or biological systems, which can, of course, vary. It should also to be understood that the terminology used herein is for the purpose of describing particular aspects of the present invention only, and is not intended to be limiting. Unless defined otherwise, all technical and scientific terms used herein have the same meaning as commonly understood by one of ordinary skill in the art to which the present invention pertains. Although any methods and materials similar or equivalent to those described herein can be used in the practice of the present invention, the preferred materials and methods are described herein. In describing and claiming the present invention, the following terminology will be used.

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

The term “construct” refers to a recombinant nucleotide sequence, generally a recombinant nucleic acid molecule, that has been generated for the purpose of the expression of a specific nucleotide sequence(s), or is to be used in the construction of other recombinant nucleotide sequences.

The term “gene” refers to a nucleic acid comprising a nucleotide sequence that encodes a polypeptide or a biologically active ribonucleic acid (RNA) such as a tRNA, shRNA, miRNA, etc. The nucleic acid can include regulatory elements (e.g., expression control sequences such as promoters, enhancers, an internal ribosome entry site (IRES)) and/or introns. A “gene product” or “expression product” of a gene is an RNA transcribed from the gene (e.g., pre- or post-processing) or a polypeptide encoded by an RNA transcribed from the gene (e.g., pre- or post-modification).

The terms “gene of interest,” “nucleotide sequence of interest” and “nucleic acid of interest” refer to any nucleotide or nucleic acid sequence that encodes a protein or other molecule that is desirable for expression in a host cell (e.g., for production of the protein or other biological molecule (e.g., an RNA product) in the target cell). The nucleotide sequence of interest is generally operatively linked to other sequences which are needed for its expression, e.g., a promoter. Further, the sequence itself may be regulatory in nature and thus of interest for expression of biologies in the target cell.

The term “infectious” in reference to a recombinant lentivirus or lentiviral particle, indicates that the lentivirus or lentiviral particle is able to enter cells and to perform at least one of the functions associated with infection by a wild-type lentivirus, e.g., release of the viral genome in the host cell cytoplasm, entry of the viral genome into the nucleus, reverse transcription, and/or integration of the viral genome into the host cell's DNA. It is not intended to indicate that the virus or viral particle is capable of undergoing replication or of completing the viral life cycle. Similarly, the term “infectivity” as used herein in reference to a recombinant lentiviral vector construct, lentivirus or lentiviral particle indicates the ability or the enhanced ability to enter cells and to perform at least one of the functions associated with infection by a wild-type lentivirus. For example, the term “enhanced infectivity” or “enhancing the infectivity” as used herein in reference to a recombinant lentiviral vector construct, lentivirus or lentiviral particle indicates the enhanced or significantly measurable increase in the ability to enter cells and to perform at least one of the functions associated with infection by a wild-type lentivirus compared to a control recombinant lentiviral vector construct, lentivirus or lentiviral particle (e.g., a recombinant lentiviral vector construct, lentivirus or lentiviral particle not comprising a GRPE element).

The term “nucleic acid” refers to polynucleotides such as DNA or RNA. Nucleic acids can be single-stranded, partly or completely, double-stranded, and in some cases partly or completely triple-stranded. Nucleic acids include genomic DNA, cDNA, mRNA, etc. Nucleic acids can be purified from natural sources, produced using recombinant expression systems and optionally purified, chemically synthesized, e.g., iRNA, siRNAs, microRNAs, and ribonucleoproteins. Where appropriate, e.g., in the case of chemically synthesized molecules, nucleic acids can comprise nucleoside analogs such as analogs having chemically modified bases or sugars, backbone modifications, etc. The term “nucleic acid sequence” as used herein can refer to the nucleic acid material itself and is not restricted to the sequence information (i.e., the succession of letters chosen among the five base letters A, G, C, T, or U) that biochemically characterizes a specific nucleic acid, e.g., a DNA or RNA molecule. A nucleic acid sequence is presented in the 5′ to 3′ direction unless otherwise indicated. The term “nucleic acid segment” is used herein to refer to a nucleic acid sequence that is a portion of a longer nucleic acid sequence.

The terms “operably linked” and “operably associated” refer to a functional relationship between two nucleic acids, wherein the expression, activity, localization, etc., of one of the sequences is controlled by, directed by, regulated by, modulated by, etc., the other nucleic acid. The two nucleic acids are said to be operably linked or operably associated or in operable association. “Operably linked” or “operably associated” can also refer to a relationship between two polypeptides wherein the expression of one of the polypeptides is controlled by, directed by, regulated by, modulated by, etc., the other polypeptide. Typically a first nucleic acid sequence that is operably linked to a second nucleic acid sequence, or a first polypeptide that is operatively linked to a second polypeptide, is covalently linked, either directly or indirectly, to such a sequence, although any effective three-dimensional association is acceptable. One of ordinary skill in the art will appreciate that multiple nucleic acids, or multiple polypeptides, may be operably linked or associated with one another.

The term “plasmid” refers to a circular nucleic acid vector. Plasmids contain an origin of replication that allows many copies of the plasmid to be produced in a bacterial or eukaryotic cell (e.g., 293T producer cell) without integration of the plasmid into the host cell DNA.

The term “promoter” as used herein refers to a recognition site of a DNA strand to which the RNA polymerase binds. The promoter forms an initiation complex with RNA polymerase to initiate and drive transcriptional activity. The complex can be modified by activating sequences termed “enhancers” or inhibitory sequences termed “silencers”.

The term “packaging” refers to the process of sequestering (or packaging) a viral genome inside a protein capsid, whereby a virion particle is formed. This process is also known as encapsidation. As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi “Ψ” sequence) needed for encapsidation of the viral genome. Thus, as used herein, the terms “packaging sequence,” “packaging signal,” “psi” and the symbol “Ψ” are used in reference to the non-coding sequence required for encapsidation of retroviral RNA strands during viral particle formation. The term includes naturally occurring packaging sequences and also engineered variants thereof. Primary packaging signals of a number of different retroviruses, including lentiviruses, are known in the art.

The term “recombinant” refers to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus or viral particle is one that comprises a recombinant nucleic acid. A recombinant cell is one that comprises a recombinant nucleic acid.

The terms “regulatory sequence” and “regulatory element” refer to a nucleic acid sequence that regulates one or more steps in the expression (particularly transcription, but in some cases other events such as splicing or other processing) of nucleic acid sequence(s) with which it is operatively linked. The terms include promoters, enhancers and other transcriptional control elements that direct or enhance transcription of an operatively linked nucleic acid. Regulatory sequences may direct constitutive expression (e.g., expression in most or all cell types under typical physiological conditions in culture or in an organism), cell type specific, lineage specific, or tissue specific expression, and/or regulatable (inducible or repressible) expression.

The term “retrovirus” refers to any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). “Retroviruses” of the invention also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

Retroviruses are RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions, appears at both the both the 5′ and 3′ ends of the viral genome. In one embodiment of the invention, the promoter within the LTR, including the 5′ LTR, is replaced with a heterologous promoter. Examples of heterologous promoters which can be used include, for example, the cytomegalovirus (CMV) promoter.

The term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

The term “hybrid” refers to a vector, LTR or other nucleic acid containing both lentiviral sequences and non-lentiviral retroviral sequences.

The term “transfection” refers to the introduction of foreign DNA into eukaryotic cells. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “transduction” refers to the delivery of a gene(s) using a viral or retroviral vector by means of viral infection rather than by transfection. In preferred embodiments, retroviral vectors are transduced by packaging the vectors into virions prior to contact with a cell.

The term “promoter/enhancer” refers to a segment of DNA which contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The term “retroviral vector” refers to a vector containing structural and functional genetic elements that are primarily derived from a retrovirus.

The term “lentiviral vector” refers to a vector containing structural and functional genetic elements outside the LTRs that are primarily derived from a lentivirus.

A retroviral vector is considered a “lentiviral vector” if at least approximately 50% of the retrovirus derived long terminal repeat, LTR (e.g., 5′LTR and/or 3′ LTR) and primary packaging sequences (e.g., Ψ) in the vector are derived from a lentivirus and/or if the LTR and primary packaging sequences are sufficient to allow an appropriately sized nucleic acid comprising the sequences to be reverse transcribed and packaged in a mammalian or avian cell that expresses the appropriate lentiviral proteins. Typically, LTR and primary packaging sequences derived from a lentivirus for use in a lentiviral vector of the invention may be at least approximately 50%, approximately 60%, approximately 70%, approximately 80%, approximately 90%, or identical to lentiviral LTR and primary packaging sequences. In certain embodiments of the invention between approximately 90 and approximately 100% of the LTR and primary packaging sequences are derived from a lentivirus. For example, the LTR and primary packaging sequences may be between approximately 90% and approximately 100% identical to lentiviral LTR and primary packaging sequences.

The term “RNAi agent” refers to an at least partly double-stranded RNA having a structure characteristic of molecules that are known in the art to mediate inhibition of gene expression through an RNAi mechanism or an RNA strand comprising at least partially complementary portions that hybridize to one another to form such a structure. When an RNA comprises complementary regions that hybridize with each other, the RNA will be said to self-hybridize. An RNAi agent includes a portion that is substantially complementary to a target nucleic acid sequence or gene. An RNAi agent optionally includes one or more nucleotide analogs or modifications. One of ordinary skill in the art will recognize that RNAi agents that are synthesized in vitro can include ribonucleotides, deoxyribonucleotides, nucleotide analogs, modified nucleotides or backbones, etc., whereas RNAi agents synthesized intracellularly, e.g., encoded by DNA templates, typically consist of RNA, which may be modified following transcription. Of particular interest herein are short RNAi agents, i.e., RNAi agents consisting of one or more strands that hybridize or self-hybridize to form a structure that comprises a duplex portion between about 15-29 nucleotides in length, optionally having one or more mismatched or unpaired nucleotides within the duplex. RNAi agents include short interfering RNAs (siRNAs), short hairpin RNAs (shRNAs), and other RNA species that can be processed intracellularly to produce shRNAs including, but not limited to, RNA species identical to a naturally occurring miRNA precursor or a designed precursor of an miRNA-like RNA.

The terms “vector” and “vector construct” refer to a nucleic acid molecule capable transferring or transporting another passenger DNA or RNA nucleic acid molecule (i.e., a sequence or gene of interest) into a host cell. For instance, either a DNA or RNA vector can be used to derive viral particles. Similarly, a cDNA copy can be made of a viral RNA genome. Alternatively, a cDNA (or viral genomic DNA) moiety can be transcribed in vitro to produce RNA. These techniques are well-known to those skilled in the art, and also are described. The transferred nucleic acid (i.e., a sequence or gene of interest) is generally linked to, e.g., inserted into, the vector nucleic acid molecule. A vector may include sequences that direct autonomous replication in a cell, or may include sequences sufficient to allow integration into host cell DNA. The vector is not a wild-type strain of a virus, inasmuch as it comprises human-made mutations or modifications. Thus, the vector typically is derived from a wild-type viral strain by genetic manipulation (e.g., by addition, deletion, mutation, insertion or other techniques known in the art) to comprise lentiviral vectors, as further described herein. In some embodiments of the present invention, the lentiviral vector constructs for use in a pharmaceutical composition (e.g., a vaccine) comprise those lentiviral vectors in which the lentiviral integrase function has been deleted and/or abrogated by site directed mutagenesis. Useful vectors include, for example, plasmids (typically DNA plasmids, but RNA plasmids are also of use), phages, cosmids, and viral vectors.

The term “viral vector” refers to either a nucleic acid molecule (e.g., a plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In particular, the terms “lentiviral vector,” “lentiviral expression vector,” etc. may be used to refer to lentiviral particles and/or lentiviral transfer plasmids of the invention as described herein. The phrase “essential lentiviral protein” as used herein refers to those viral protein(s), other than envelope protein, that are required for the lentiviral life cycle. Essential lentiviral proteins may include those required for reverse transcription and integration and for the packaging (e.g., encapsidation) of a retroviral genome.

The terms “subject,” “patient,” “individual,” and “host” used interchangeably herein, refer to a mammal, including, but not limited to, murines, felines, simians, humans, mammalian farm animals, mammalian sport animals, and mammalian pets. The term includes mammals that are infected with as well as those that are susceptible to infection by an immunodeficiency virus. In certain embodiments, the term refers to a human infected with HIV.

“HIV” is used herein to refer to the human immunodeficiency virus. It is recognized that the HIV virus is an example of a hyper-mutable retrovirus, having diverged into two major subtypes (HIV-1 and HIV-2), each of which has many subtypes. In some embodiments, a human subject is infected with the HIV-1 subtype.

As used herein, the term “viral infection” describes a diseased state in which a virus invades healthy cells, uses the cell's reproductive machinery to multiply or replicate and ultimately lyse the cell resulting in cell death, release of viral particles and the infection of other cells by the newly produced progeny viruses. Latent infection by certain viruses, e.g., HIV-1, is also a possible result of viral infection.

Embodiments described herein relate to an HIV based vaccine system that includes an HIV multivalent vector with diverse HIV envelope genes that are produced through forced recombination of the env region using of yeast-based cloning methods. The system can rapidly produce HIV envelope clones. The produced viruses are similar to wild-type HIV virus, and can mimic the entry process, expose hidden epitopes, complete reverse transcription, integrate into host chromosome, and continuously produce HIV envelope proteins, to elicit both humoral and cellular immune responses. In some embodiments, the produced env recombinants only contain break points in the gp41 region. In other embodiments, the vaccine system can be used to generate and/or screen for broadly neutralizing anti-HIV antibodies.

In some embodiments, the multivalent vaccines can be controlled to contain as few as 10 to greater than 1,000 unique variants where the recombination breakpoints in a quarter of the variant occurs within a codon to generate a nonsynonymous but functional amino acid substitution. The degree of functional heterogeneity of this heterologous subtype vaccine can be designed to be greater than in the autologous, heterogeneous HIV-1 vaccine. This diversity of the heterologous vaccine can be sufficient as a therapeutic vaccine.

In some embodiments, the HIV based vaccine system can include at least one recombinant HIV-1 particle prepared from an HIV-1 RNA sample obtained from a subject with HIV-1. HIV-1 particles described herein can include viral Gag, Pol, and Env proteins and a viral genome that comprises a nucleic acid including a GRPE element and sequences sufficient for reverse transcription and packaging may be used to deliver transgenic material to a target cell. The viral genome may further comprise regulatory sequences sufficient to promote transcription of an operably linked sequence of interest. The recombinant HIV-1 particles are replication-defective, i.e., the viral genome does not encode functional forms of all the proteins necessary for the infective cycle. For example, sequences encoding a structural protein or a protein required for replication may be mutated or disrupted or may be partly or completely deleted and/or replaced by a different nucleic acid sequence, e.g., a nucleic acid sequence of interest that is to be introduced into a target cell. However, sequences required for reverse transcription, integration, and packaging are typically functional.

The HIV-1 RNA can include an HIV-1 protein coding sequence that comprises HIV-1 envelope (env), gag and/or pol protein coding sequences and combinations thereof. In some embodiments the HIV-1 protein coding sequence is an HIV-1 gag/pol protein or an env coding sequence.

Any HIV-1 gag (group-specific antigen) protein coding sequence derived from an HIV-1 virus of a subject can be used. Exemplary gag protein coding sequences derived from an HIV-1 virus include gag protein coding sequences for the precursor gag polyprotein which is processed by viral protease during maturation to MA (matrix protein, p17); CA (capsid protein, p24); SP1 (spacer peptide 1, p2); NC (nucleocapsid protein, p7); SP2 (spacer peptide 2, p1) and P6 protein.

Any HIV-1 pol protein coding sequence derived from an HIV-1 virus of a subject can be used. Exemplary pol protein coding sequences derived from an HIV-1 virus include pol protein coding sequences for viral enzymes reverse transcriptase (RT) and RNase integrase (IN), and HIV protease (PR).

Any HIV-1 envelope protein coding sequence derived from an HIV-1 virus of a patient that mediates membrane fusion can be used. As used herein, the term “HIV-1 envelope protein” refers to a full-length protein, fragment, analog, or derivative thereof. The HIV-1 envelope protein coding sequence derived from an HIV-1 virus of a patient may be a sequence coding a surface glycoprotein.

HIV-1 envelope protein coding sequences useful in the present method can include, but are not limited to HIV-1 envelope protein coding sequences encoding surface proteins from a number of different HIV-1 groups. Exemplary HIV-1 groups include both the “major” group (i.e., the M group) and the minor groups O, N and P. An HIV-1 envelope protein useful in the present method can also include subgroups, or clades, of HIV-1 groups known in the art.

In some embodiments, the HIV-1 envelope protein coding sequences encode surface proteins from a patient infected with a HIV-1 group M subtype B variant. Non-limiting examples of subtype B HIV-1 variants can include HIV-1B-92BR014, HIV-1B-92TH593, HIV-1B-92US727, and HIV-1B-92US076.

In some embodiments, the HIV-1 envelope protein coding sequences encode surface proteins from a patient infected with a HIV-1 group M non-subtype B variant. Non-B HIV-1 group M variants can include the clades or subtypes A, C, D, F, G, H, J, K, N and circulating recombinant forms derived from recombination between viruses of different subtypes. Non-limiting examples of non-B HIV-1 subtypes include three subtype A (HIV-1A-93RW024, HIV-1A-92UG031, and HIV-1A-92UG029), four subtype C (HIV-1C-96USNG58, HIV-1C-93MW959, HIV-1C-98IN022, and HIV-1C-92BR025), five subtype D (HIV-1D-92UG021, HIV-1D-92UG024, HIV-1D-94UG114, HIV-1D-92UG038, and HIV-1D-93UG065), two subtype F (HIV-1F-93BR20 and HIV-1F-93BR29), two subtype G (HIV-1G-RU132 and HIV-1G-RU570), and six circulating recombinant forms (HIV-1AE-CMU02, HIV-1AE-CMU06, HIV-1AE-92TH021, HIV-1AE-93TH051, HIV-1AE-95TH001, and HIV-1BF-93BR029).

Samples obtained from a subject can include blood samples. In certain embodiments, the sample is a blood plasma sample. In some embodiments, a blood sample can be collected from a patient and plasma samples can be processed for immediate use. Alternatively, a processed plasma sample can be stored at −80° C. for analysis at a later time.

In some embodiments, a blood sample from the HIV-infected patient has a viral load ranging from about <50 to about 10,000 copies of viral RNA/ml. In some embodiments, the viral load can range from about 1000 to about 10,000 copies/ml. In certain embodiments, a blood sample from the HIV-infected patient has a viral load ≧1,000 copies/ml.

In some embodiments, plasma viral HIV-1 RNA coding for at least one HIV-1 coding sequence can be purified from pelleted virus particles using well known methods. In one example, plasma viral HIV-1 RNA can be purified from pelleted virus particles by centrifuging one milliliter of a patient's plasma at 20,000 g×60 min at 4° C. using a QIAamp Viral RNA Mini Kit (Qiagen).

Once the plasma HIV-1 RNA is obtained and purified it can be reverse transcribed into HIV-1 cDNA. A representative protocol for the preparation of HIV-1 cDNA from purified HIV-1 RNA includes adding 10 μl of the backward (BWD) primer EXT TAT REC CON BWD 13 having SEQ ID NO: 47 to 7.25 μl of DEPC-treated H₂O, 2.0 μl of RT Buffer 10× and 2.0 μl of 10 mM dNTP mix. This mixture is further added to 5 μl of purified HIV-1 RNA and 4 μl of PCR water and incubated at 88° C. for 1 minute, 65° C. for 10 minutes, and then 25° C. for 5 min. The resulting mixture is then kept at room temperature. Next, 2.0 μl of 100 mM DTT, 0.25 μl (10 U) RNase inhibitor, and 0.5 μl AccuScript High-Fidelity Reverse Transcriptase (AccuRT) (STRATAGENE) is added individually to the mixture. The mixture is then incubated at 42° C. for 90 min, heat inactivated at 70° for 15 minutes, and chilled and held at 4° for PCR amplification. Alternatively, the mixture can be frozen at −20° for later amplification.

A portion of the HIV-1 cDNA corresponding to an HIV-1 env, gag and/or pol protein coding sequence derived from a patient with HIV-1 can be amplified using a PCR assay where the patient derived HIV-1 cDNA acts as a template. In some embodiments, PCR amplification of the envelope protein coding sequence amplifies a portion of the patient's env gene from gp120 up to Tat exon 2. In some embodiments, PCR amplification of the envelope protein coding sequence amplifies a 2302 nt fragment of the HIV-1 env gene, including the entire surface glycoprotein (gp120) and most of the transmembrane glycoprotein (gp41) such that recombinant particles produced from by forced recombination only contain breakpoints in the gp41 region.

The use of both external and nested env, gag and/or pol gene specific primers (i.e., a nested PCR) can be employed to amplify an HIV-1 envelope protein coding sequence of patient derived HIV-1 cDNA.

Amplified PCR products corresponding to a patient derived HIV-1 envelope, gag and/or pol protein coding sequence (i.e., the patient derived amplicon) can then be purified. For example, PCR cDNA products corresponding to the gp120/gp41-coding regions of an HIV-1 envelope protein derived from a patient can be purified using a QIAquick PCR Purification Kit (QIAGEN).

At least a first patient derived HIV-1 protein coding sequence and a second patient derived HIV-1 protein coding sequence can be introduced into first and second expression constructs using a yeast based homologous recombination/gap repair method.

An expression construct can include a vector, such as a plasmid. A suitable vector includes at least one origin of replication, a region of the DNA that is substantially identical to the primer binding site (pbs) of HIV-1, a selectable gene replacing at least a portion of the env, gag, and/or pol gene of HIV-1, and a region of DNA that is substantially identical to the 3′ end of the long terminal repeat region of HIV. By “substantially identical”, it is meant that the regions have sufficient homology with the named segments of DNA as to be able to hybridize under stringent conditions.

A suitable vector can also comprise a partial retrovirus genome, specifically; a vector can include a near full length (nfl) HIV-1 genome devoid of the 5′ LTR. Lack of a 5′ LTR allows the HIV-1 genome to be located precisely in front of the CMV promoter in the vector such that transcription would be initiated at the first nucleotide of the primer binding site. Cloning the HIV-1 sequence in this way could not be performed with restriction enzymes but can be performed by yeast recombination. In addition a vector devoid of the HIV-1 5′ LTR is unable to produce infectious virus. Vectors can include the essential elements for plasmid growth in bacteria and for HIV-1 expression in human cells. In addition, the yeast-based cloning system allows for cloning into SIVmac, SIVcpz, and SHIV molecular clones.

Vectors can include a sequence corresponding to a near full length HIV-1 backbone. In some embodiments, the near full length HIV-1 backbone includes a HIV-1 Group M subtype B backbone (e.g., HIV-1_{NL4-3}). In certain embodiments, the vector can recombine with not only homologous env, gag and/or pol protein coding sequences derived from patients infected with Group M subtype B wild-type and multidrug resistant strains of HIV-1 but also from sequences derived from patients infected with other non-B HIV-1 group M subtypes. Therefore, in some embodiments the near full length HIV-1 backbone of a vector can include a minor HIV-1 group backbone and a method of the present invention can be used to determine HIV-1 co-receptor tropism in a patient infected with a minor HIV-1 group strain. Exemplary minor HIV-1 group backbones can include Group N, Group 0 and Group P strains near full length HIV-1 backbones.

In certain embodiments, the near full length HIV-1 yeast-based vector pREC nfl HIV-1 Δenv/URA3 is employed. pREC nfl HIV-1 Δenv/URA3 contains the selection marker URA3. URA3 encodes the orotidine-5′-phosphate decarboxylase protein involved in the biosynthesis of uracil. To prepare pREC nfl HIV-1 Δenv/URA3, URA3 is recombined in yeast to replace a section of the env gene in the pREC nfl HIV-1 vector resulting in a vector having the pREC nfl HIV-1 sequence except with a URA3 gene inserted into and replacing a portion of the envelope gene. Successful recombinants may be selected by growing the yeast transformed with the URA3 and the pREC nfl HIV-1 on uracil-deficient media. In certain embodiments, at least a portion of the 5′ and 3′ ends of the pREC nfl HIV-1 env gene remain so as to permit further recombination.

In addition to URA3, a pREC nfl HIV-1 Δenv/URA3 vector can also include a yeast transformation selection marker gene that does not replace a portion of the envelope gene (e.g., LEU2 or TRP1).

Expression constructs can be made, for example, by replacing various portions of the HIV-1 env, gag and/or pol gene in the pREC nfl HIV-1 vector with a selectable marker such as URA3. URA3 may be inserted into the pREC nfl HIV-1 vector at different sites for replacement of the gp120/gp41, the gp120, or V3 coding sequence in the HIV-1 envelope gene, for example. In some embodiments, expression constructs are provided that only include break points in the gp41 region. A list of near full length HIV-1 isolates containing a URA3 substitution for use in the present invention is provided in Table 1.

TABLE 1
pREC nfl HIV-1 vectors with various
coding region replacements with URA3

| | Location of Deletion | |
|---|---|---|
| pREC-_{NFL-HIV-1} Deletions | in NL4-3 | Size of Deletion |
| Δenv\URA3 | 6221-8785 | 2565 |
| Δenv-s\URA3 | 6221-8264 | 2043 |
| Δenv gp120\URA3 | 6221-7747 | 1527 |
| Δenv gp120 v1/v2\URA3 | 6611-6802 | 192 |
| Δenv gp120 v3\URA3 | 7100-7207 | 108 |
| Δenv gp120 v4/v5\URA3 | 7368-7627 | 260 |
| Δenv gp41\URA3 | 7748-8785 | 1038 |
| Δenv gp41-s\URA3 | 7748-8264 | 517 |

To insert a purified HIV-1 protein coding sequence derived from a patient and replace a selectable gene encoded by the vector, a yeast strain (e.g., Strain BY4727) may be transformed with either linearized or non-linearized pREC_nfl_HIV-1Δproteome/URA3, using a lithium acetate technique for example, along with the purified HIV-1 protein coding cDNA sequence derived from a patient. The patient derived cDNA recombines with the remaining portions of the env, gag and/or pol gene flanking the URA3 gene in pREC nfl HIV-1 Δproteome/URA3. The resulting recombinants contain a near full length HIV-1 sequence from the NL4-3 HIV-1 strain, with a patient-derived env, gag and/or pol gene or gene fragment replacing the env, gag and/or pol gene of NL4-3.

In some embodiments, PCR products spanning the gp120/gp41-coding region of HIV-1 derived from a patient are introduced via yeast homologous recombination into a pRECnfl ΔEnv/URA3 vector. The pRECnfl-TRPΔEnv/URA3 vector includes a near-full length HIV-1 genome where a yeast uracil biosynthesis (URA3) gene has replaced the native gp120/gp41 HIV-1 coding sequence. Following successful yeast homologous recombination of the gp120/gp41-coding region of HIV-1 derived from a patient and the pRECnfl-TRPΔEnv/URA3 vector, the vector construction expresses all HIV-1 coding regions, that is, all genes corresponding to the HIV-1_{NFL4-3} strain used as backbone in the vector plus the patient-derived HIV-1 envelope protein coding sequence; however, it is unable to produce infectious virus since it is missing the 5′ LTR region. In another embodiment, PCR products spanning the gp120/gp41-coding region of HIV-1 derived from a patient are introduced via yeast homologous recombination into a pREC_SIN_HIV-1ΔEnv/URA3 vector.

In another exemplary embodiment, PCR products spanning both the gag and pol (i.e., gag/pol) coding region of HIV-1 derived from a patient are introduced via yeast homologous recombination into a pRECnfl-Δgag-pol/URA3 vector (e.g., pREC_exp_HIV-1 Δgag-pol/URA3). The pRECnfl-Δgag-pol/URA3 vector includes a near-full length HIV-1 genome where a yeast uracil biosynthesis (URA3) gene has replaced at least a portion of the native gag/pol HIV-1 coding sequence.

Yeast colonies containing a recombined sequence in the pREC nfl HIV-1 vectors, for example, where a URA3 gene has been replaced by the HIV-1 env, gag and/or pol protein coding sequence derived from a patient, may be selected on plates containing a selection agent, such as CMM-Leu+5-Fluoro-1,2,3,6-Tetrahydro-2,6-Dioxo-4-Pyrimidine Carboxylic Acid (FOA). FOA is converted to the toxic substrate 5-fluorouracil by the URA3 gene product, orotidine-5′-phosphate decarboxylase. FOA-resistant yeast including the newly recombined expression construct can then be grown in yeast complete minimal medium. In one example over 95%-98% of all yeast colonies following transfection harbor vectors with the correct insert and in the correct reading frame due to highly specific recombination.

Organisms other than yeast may also be utilized to provide homologous recombination. For example, the bacterial strains TB10-pyrF287 and TB10ΔpyrF can also be used for recombination of patient derived HIV-1 envelope, gag and/or pol protein coding sequences into the pREC nfl HIV-1 plasmids. TB10ΔpyrF strain genotype is nad::Tn10/pλ-Δcro-bro tetr pyrF. TB10ΔpyrF287 strain genotype is nad::Tn10/pλ-Δcro-bro tetr pyrF287. These strains express λ bet, gam, and exo for hyper-recombination. Additionally, pyrF is the homolog to URA3. Deleting and mutating pyrF in TB10-pyrF287 and TB10λpyrF can allow URA3 plasmids to be used for selection. This will allow the same plasmids to be currently used in the yeast system to be used in the bacterial system.

Following homologous recombination, the first and second expression constructs can then be extracted and purified from the organisms providing recombination. For example, recombined pREC nfl HIV-1 vectors including the HIV-1 envelope, gag and/or pol protein coding sequence(s) derived from a patient can be purified from the entire number of yeast colonies. In some embodiments, an expression construct is extracted and purified from about 200 to greater than 1000 individual yeast colonies.

The purified expression constructs can then be transformed into bacteria (e.g., E. coli) for plasmid vector propagation. In an exemplary embodiment, Electrocomp TOP10 E. coli bacteria cells (Invitrogen) are transformed with purified recombined pREC nfl HIV-1 vectors. Plasmid DNA, once purified from the bacteria, can be stored at −80° C. until further use. In an alternative embodiment, bacterial colonies can be transformed with crude yeast extract without the purification step. In some embodiments, transformed bacterial colonies can be screened for the env, gag and/or pol insert and absence of the URA3 gene using well known methods.

A cell can then be transfected with the first and second expression construct. In some embodiments, a cell is transfected with two, three, or four expression constructs each including at least one HIV-1 coding sequences derived from a subject and/or an HIV-1 backbone sequence. Methods of transfecting and expressing genes in mammalian cells are known in the art. Transducing cells with viral vectors can involve, for example, incubating vectors with cells within the viral host range under conditions and concentrations necessary to cause transduction. See, e.g., Methods in Enzymology, vol. 185, Academic Press, Inc., San Diego, Calif. (D. V. Goeddel, ed.) (1990) or M. Krieger, Gene Transfer and Expression—A Laboratory Manual, Stockton Press, New York, N.Y.; and Muzyczka (1992) Curr Top. Microbiol. Immunol. 158: 97-129, and references cited in each. The culture of cells, including cell lines and cultured cells from tissue samples is well known in the art. Freshney (Culture of Animal Cells, a Manual of Basic Technique, Third edition Wiley-Liss, New York (1994)) provides a general guide to the culture of cells.

An expression construct for use in a method of the invention can include a vector, such as a plasmid. Suitable vectors for use in eukaryotic and prokaryotic cells are known in the art and are commercially available or readily prepared by a skilled artisan. Additional vectors can also be found, for example, in CURRENT PROTOCOLS IN MOLECULAR BIOLOGY (Ausubel, F. M., et al., eds. 2000) and Sambrook et al., “Molecular Cloning: A Laboratory Manual,” 2nd ED. (1989), the teachings of which are incorporated herein by reference. In some embodiments, transformation of a cell with a reporter molecule fragment sequence can be achieved using calcium phosphate, DEAE-dextran, electroporation, cationic lipid reagents, or any other convenient technique known in the art.

In some embodiments, the expression vectors can be mammalian expression vectors including a promoter operably linked to a reporter molecule fragment expression sequence. For example, a CMV promoter-based vector can be used. Human cytomegalovirus (CMV) promoter regulatory region drives constitutive protein expression. In certain embodiments, the expression vector is a pCMV_cplt expression vector. The pCMV_cplt expression vector can be constructed by PCR-amplifying the cytomegalovirus (CMV) sequence from pcDNA3.1zeo/CAT (Invitrogen). The TOPO vector was then subjected to digestion by MLUI and BstXI to generate a CMV promoter-driven R, U5 and gag fragment. The resulting fragment is cloned back into the pcDNA3.Izeo/CAT backbone to generate the pCMV_cplt vector.

Any cell can be transfected with the vectors. The cell can be human or nonhuman. The cell can be freshly isolated (i.e., primary) or derived from a short term- or long term-established cell line. In one embodiment, the first is a eukaryotic cell, where the eukaryotic cell is a cell that can be grown in culture, using standard laboratory procedures and media well known to those of skill in the art. The cell may be any cell that is not susceptible to toxic effects of chronically expressing viral proteins and that permit cell surface expression of such proteins. In some embodiments, the cell is a cell that does not express complete complementary cell surface receptors or co-receptors for the expressed patient-derived HIV-1 env coded protein and therefore will not undergo fusion with itself.

Exemplary biological cell lines include NIH-3T3 murine fibroblasts, quail QT6 cells, canine Cf2Th thymocytes, Mv1 Lu mink lung cells, Sf9 insect cells, primary T-cells, human T-cell lines (e.g., H-9), U-87 MG glioma, SCL1 squamous cell carcinoma cells, CEM, HeLa epithelial carcinoma, Chinese hamster ovary (CHO) cell, SF33 cell and HEK293T cell. Such cell lines are described, for example, in the Cell Line Catalog of the American Type Culture Collection (ATCC, Rockville, Md.). In one embodiment, the first cell is a HeLa epithelial carcinoma cell or a human HEK293T cell.

In one embodiment, a cell stably expresses the recombinant HIV-1 particles and secretes it from the cell. For example, a cell may comprise a coding sequence for the HIV-1 particles stably integrated into its genome in a manner such that it is expressed in the cell and directed to the cell surface where it is secreted into the surrounding media. Once secreted into the media, the recombinant HIV-1 particles can be harvested for therapeutic use as described below. In some embodiments, the recombinant HIV-1 particles are harvested about 48 to about 72 hours post-transfection. Harvested the recombinant HIV-1 particles can then be purified for example, through the use of sucrose-cushion centrifugation, and then quantified for capsid/p24 content.

The HIV-1 particles secreted by the cell is a defective HIV-1 particle including env, gag and pol coded proteins in the correct stoichiometry and is morphologically indistinguishable from a wild type HIV-1. For example, the cloning system described herein allows for the pREC HIV-1 nfl plasmid upon 293T transfections to produce vectors that lack 5′LTR and as such cannot initiate reverse transcription, lack a functional reverse transcriptase enzyme, lack genomic RNA due to deletion of Ψ packaging element, contains full complement of HIV-1 proteins in the correct stoichmetry, and are dead but morphologically identical to wild type.

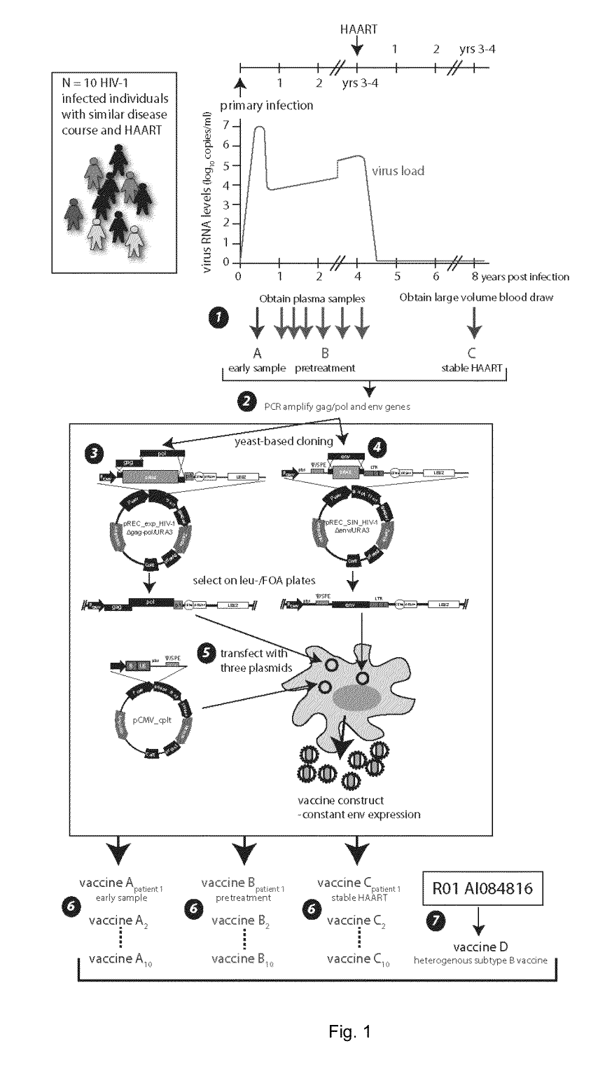

In another exemplary embodiment, a 293T cell is transfected with a pREC_exp-_HIV-1 Δgag-pol/URA3 and a pREC_SIN_HIV-1ΔEnv/URA3 derived from a subject having HIV-1, and a pCMV_cplt expression vector expressing defective genomic RNA. As shown in FIG. 1a, the transfected cell constantly expresses the resulting vaccine construct. The separate Gag-Pol vector will support virus production but the mRNA cannot be preferentially encapsidated or if randomly encapsidated at low frequencies, will not support reverse transcription/proviral DNA synthesis.

Once a nucleic acid is incorporated into a cell as provided herein, the cell can be maintained under suitable conditions for constant expression of the recombinant HIV-1 particles. Generally, the cells are maintained in a suitable buffer and/or growth medium or nutrient source for growth of the cells and expression of the gene product(s). Exemplary growth medium can include, but is not limited to, DMEM medium/L-glutamine (GIBCO; CELLGRO; MEDIATECH) supplemented with FBS (CELLGRO), penicillin/streptomycin (GIBCO), puromycin and G418 (MEDIATECH).

As described in more detail below, the recombinant HIV-1 particles can be used to form a therapeutic composition, such as a vaccine or pharmaceutical composition. The vaccine can include a therapeutically effective amount of the replication defective recombinant HIV-1 particles and a pharmaceutically acceptable carrier.

While it is possible that the vaccine can comprise the replication defective recombinant HIV-1 particles in a pure or substantially pure form, it will be appreciated that the vaccine can additionally or optionally include the replication defective recombinant HIV-1 particles and a pharmaceutically acceptable carrier or other therapeutic agent. For example, the pharmaceutically acceptable carrier can include a physiologically acceptable diluent, such as sterile water or sterile isotonic saline. As used herein, the term “pharmaceutically acceptable carrier” can refer to any and all solvents, dispersion media, coatings, antibacterial and antifungal agents, isotonic and absorption delaying agents, and the like.

Additional components that may be present with the vaccine can include adjuvants, preservatives, chemical stabilizers, and/or other proteins. Typically, stabilizers, adjuvants, and preservatives are optimized to determine the best formulation for efficacy in a subject. Exemplary preservatives can include, but are not limited to, chiorobutanol, potassium sorbate, sorbic acid, sulfur dioxide, propyl gallate, the parabens, ethyl vanillin, glycerin, phenol, and parachiorophenol. Suitable stabilizing ingredients can include, for example, casamino acids, sucrose, gelatin, phenol red, N—Z amine, monopotassium diphosphate, lactose, lactalbumin hydrolysate, and dried milk. Other examples of pharmaceutically acceptable carriers are known in the art and described below.

A vaccine comprising the replication defective recombinant HIV-1 particles can be used either prophylactically or therapeutically. When provided prophylactically, the vaccine can be provided in advance of any evidence of an active HIV infection and thereby attenuate or prevent HIV infection. For example, a human at high risk for HIV infection can be prophylactically treated with a vaccine comprising the replication defective recombinant HIV-1 particles and a pharmaceutically acceptable carrier. When provided therapeutically, the vaccine can be used to enhance a subject's own immune response to the antigens present as a result of HIV infection. It will be appreciated that the replication defective recombinant HIV-1 particles can be conjugated with one or more lipoproteins, administered in liposomal form, or with an adjuvant.

Another aspect of the application can include a method for inducing an immune response against HIV or an HIV epitope in a subject. The method can include administering to the subject an effective amount of the replication defective recombinant HIV-1 particles that elicits the immune response and thereby prevents or inhibits HIV infection in the subject.

Inhibiting a viral infection can refer to inhibiting the onset of a viral infection, inhibiting an increase in an existing viral infection, or reducing the severity of the viral infection. In this regard, one of ordinary skill in the art will appreciate that while complete inhibition of the onset of a viral infection is desirable, any degree of inhibition of the onset of a viral infection is beneficial. Likewise, one of ordinary skill in the art will appreciate that while elimination of viral infection is desirable, any degree of inhibition of an increase in an existing viral infection or any degree of a reduction of a viral infection is beneficial.

Inhibition of a viral infection can be assayed by methods known in the art, such as by assessing viral load. Viral loads can be measured by methods known in the art, such as by using PCR to detect the presence of viral nucleic acids or antibody-based assays to detect the presence of viral protein in a sample (e.g., blood) from a subject. Alternatively, the number of CD4+ T cells in a viral-infected subject can be measured. A treatment that inhibits an initial or further decrease in CD4+ T cells in a viral-infected subject, or that results in an increase in the number of CD4+ T cells in a viral-infected subject, for example, may be considered an efficacious or therapeutic treatment.

Optimal dosages to be administered may be readily determined by those skilled in the art, and will vary with the particular compound used, the strength of the preparation, the mode of administration, and the advancement of the disease condition. In addition, factors associated with the particular patient being treated, including patient age, weight, diet and time of administration, will result in the need to adjust dosages.

In some embodiments, a pharmaceutical composition including the replication defective recombinant HIV-1 particles derived from the subject described herein can be administered in combination with one or more additional activators of latent HIV expression. In certain embodiments, such a combination can synergistically enhance reactivation of latently infected cell populations of cells compared to either agent alone.

In some embodiments, a pharmaceutical composition administered to a subject includes a therapeutically effective amount of the replication defective recombinant HIV-1 particles, and another therapeutic agent useful in the treatment of HIV infection, such as a component used for HAART or immunotoxins.

As noted above, compositions described herein may be combined with one or more additional therapeutic agents useful in the treatment of HIV infection. It will be understood that the scope of combinations of the compounds of this invention with HIV/AIDS antivirals, immunomodulators, anti-infectives or vaccines is not limited to the following list, and includes in principle any combination with any pharmaceutical composition useful for the treatment of AIDS. The HIV/AIDS antivirals and other agents will typically be employed in these combinations in their conventional dosage ranges and regimens as reported in the art.

Examples of antiviral agents include (but not restricted) ANTIVIRALS Manufacturer (Tradename and/or Drug Name Location) Indication (Activity): abacavir GlaxoSmithKline HIV infection, AIDS, ARC GW 1592 (ZIAGEN) (nRTI); 1592U89 abacavir+GlaxoSmithKline HIV infection, AIDS, ARC (nnRTI); lamivudine+(TRIZIVIR) zidovudine acemannan Carrington Labs ARC (Irving, Tex.) ACH 126443 Achillion Pharm. HIV infections, AIDS, ARC (nucleoside reverse transcriptase inhibitor); acyclovir Burroughs Wellcome HIV infection, AIDS, ARC, in combination with AZT AD-439 Tanox Biosystems HIV infection, AIDS, ARC AD-519 Tanox Biosystems HIV infection, AIDS, ARC adefovir dipivoxil Gilead HIV infection, AIDS, ARC GS 840 (RTI); AL-721 Ethigen ARC, PGL, HIV positive, (Los Angeles, Calif.), AIDS alpha interferon GlaxoSmithKline Kaposi's sarcoma, HIV, in combination w/Retrovir AMD3100 AnorMed HIV infection, AIDS, ARC (CXCR4 antagonist); amprenavir GlaxoSmithKline HIV infection, AIDS, 141 W94 (AGENERASE) ARC (PI); GW 141 VX478 (Vertex) ansamycin Adria Laboratories ARC LM 427 (Dublin, Ohio) Erbamont (Stamford, Conn.) antibody which neutralizes; Advanced Biotherapy AIDS, ARC pH labile alpha aberrant Concepts (Rockville, Interferon Md.) AR177 Aronex Pharm HIV infection, AIDS, ARC atazanavir (BMS 232632) Bristol-Myers-Squibb HIV infection, AIDS, ARC (ZRIVADA) (PI); beta-fluoro-ddA Nat'l Cancer Institute AIDS-associated diseases BMS-232623 Bristol-Myers Squibb/HIV infection, AIDS, (CGP-73547) Novartis ARC (PI); BMS-234475 Bristol-Myers Squibb/HIV infection, AIDS, (CGP-61755) Novartis ARC (PI); capravirine Pfizer HIV infection, AIDS, (AG-1549, S-1153) ARC (nnRTI); CI-1012 Warner-Lambert HIV-1 infection cidofovir Gilead Science CMV retinitis, herpes, papillomavirus curdlan sulfate AJI Pharma USA HIV infection cytomegalovirus immune MedImmune CMV retinitis globin cytovene Syntex sight threatening CMV ganciclovir peripheral CMV retinitis delavirdine Pharmacia-Upjohn HIV infection, AIDS, (RESCRIPTOR) ARC (nnRTI); dextran Sulfate Ueno Fine Chem. Ind. AIDS, ARC, HIV Ltd. (Osaka, Japan) positive asymptomatic ddC Hoffman-La Roche HIV infection, AIDS, ARC (zalcitabine, (HIVID) (nRTI); dideoxycytidine dd1 Bristol-Myers Squibb HIV infection, AIDS, ARC; Dideoxyinosine (VIDEX) combination with AZT/d4T (nRTI) DPC 681 & DPC 684 DuPont HIV infection, AIDS, ARC (PI) DPC 961 & DPC 083 DuPont HIV infection AIDS, ARC (nnRTRI); emvirine Triangle Pharmaceuticals HIV infection, AIDS, ARC (COACTINON) (non-nucleoside reverse transcriptase inhibitor); EL10 Elan Corp, PLC HIV infection (Gainesville, Ga.) efavirenz DuPont HIV infection, AIDS, (DMP 266) (SUSTIVA) ARC (nnRTI); Merck (STOCRIN) famciclovir Smith Kline herpes zoster, herpes simplex emtricitabine Triangle Pharmaceuticals HIV infection, AIDS, ARC FTC (COVIRACIL) (nRTI); Emory University emvirine Triangle Pharmaceuticals HIV infection, AIDS, ARC (COACTINON) (non-nucleoside reverse transcriptase inhibitor); HBY097 Hoechst Marion Roussel HIV infection, AIDS, ARC (nnRTI); hypericin VIMRx Pharm. HIV infection, AIDS, ARC recombinant human; Triton Biosciences AIDS, Kaposi's sarcoma, interferon beta (Almeda, Calif.); ARC interferon alfa-n3 Interferon Sciences ARC, AIDS indinavir; Merck (CRIXIVAN) HIV infection, AIDS, ARC, asymptomatic HIV positive, also in combination with AZT/ddI/ddC (PI); ISIS 2922 ISIS Pharmaceuticals CMV retinitis JE2147/AG1776; Agouron HIV infection, AIDS, ARC (PI); KNI-272 Nat'l Cancer Institute HIV-assoc. diseases lamivudine; 3TC Glaxo Wellcome HIV infection, AIDS, (EPIVIR) ARC; also with AZT (nRTI); lobucavir Bristol-Myers Squibb CMV infection; lopinavir (ABT-378) Abbott HIV infection, AIDS, ARC (PI); lopinavir+ritonavir Abbott (KALETRA) HIV infection, AIDS, ARC (ABT-378/r) (PI); mozenavir AVID (Camden, N.J.) HIV infection, AIDS, ARC (DMP-450) (PI); nelfinavir Agouron HIV infection, AIDS, (VIRACEPT) ARC (PI); nevirapine Boeheringer HIV infection, AIDS, Ingleheim ARC (nnRTI); (VIRAMUNE) novapren Novaferon Labs, Inc. HIV inhibitor (Akron, Ohio); pentafusaide Trimeris HIV infection, AIDS, ARC T-20 (fusion inhibitor); peptide T Peninsula Labs AIDS octapeptide (Belmont, Calif.) sequence PRO 542 Progenics HIV infection, AIDS, ARC (attachment inhibitor); PRO 140 Progenics HIV infection, AIDS, ARC (CCR5 co-receptor inhibitor); trisodium Astra Pharm. Products, CMV retinitis, HIV infection, phosphonoformate Inc other CMV infections; PNU-140690 Pharmacia Upjohn HIV infection, AIDS, ARC (PI); probucol Vyrex HIV infection, AIDS; RBC-CD4Sheffield Med. Tech HIV infection, AIDS, (Houston Tex.) ARC; ritonavir Abbott HIV infection, AIDS, (ABT-538) (RITONAVIR) ARC (PI); saquinavir Hoffmann-LaRoche HIV infection, AIDS, (FORTOVASE) ARC (PI); stavudine d4T Bristol-Myers Squibb HIV infection, AIDS, ARC didehydrodeoxy-(ZERIT.) (nRTI); thymidine T-1249 Trimeris HIV infection, AIDS, ARC (fusion inhibitor); TAK-779 Takeda HIV infection, AIDS, ARC (injectable CCR5 receptor antagonist); tenofovir Gilead (VIREAD) HIV infection, AIDS, ARC (nRTI); tipranavir (PNU-140690) Boehringer Ingelheim HIV infection, AIDS, ARC (PI); TMC-120 & TMC-125 Tibotec HIV infections, AIDS, ARC (nnRTI); TMC-126 Tibotec HIV infection, AIDS, ARC (PI); valaciclovir GlaxoSmithKline genital HSV & CMV infections virazole Viratek/ICN (Costa asymptomatic HIV positive, ribavirin Mesa, Calif.) LAS, ARC; zidovudine; AZT GlaxoSmithKline HIV infection, AIDS, ARC, (RETROVIR) Kaposi's sarcoma in combination with other therapies (nRTI); [PI=protease inhibitor nnRTI=non-nucleoside reverse transcriptase inhibitor NRTI=nucleoside reverse transcriptase inhibitor]

The additional therapeutic agent may be used individually, sequentially, or in combination with one or more other such therapeutic agents described herein (e.g., a reverse transcriptase inhibitor used for HAART, a protease inhibitor used for HAART, an HIV-1 protein derived from the subject and/or an activator of latent HIV expression). Administration to a subject may be by the same or different route of administration or together in the same pharmaceutical formulation.

According to this embodiment, a composition comprising the replication defective recombinant HIV-1 particles may be coadministered with any HAART regimen or component thereof. The current standard of care using HAART is usually a combination of at least three nucleoside reverse transcriptase inhibitors and frequently includes a protease inhibitor, or alternatively a non-nucleoside reverse transcriptase inhibitor. Subjects who have low CD4+ cell counts or high plasma RNA levels may require more aggressive HAART. For subjects with relatively normal CD4+ cell counts and low to non-measurable levels of plasma HIV RNA over prolonged periods (i.e., slow or non-progressors) may require less aggressive HAART. For antiretroviral-naive subject who are treated with initial antiretroviral regimen, different combinations (or cocktails) of antiretroviral drugs can be used.

Thus, in some embodiments, a pharmaceutical composition comprising the replication defective recombinant HIV-1 particles may be coadministered to the subject with a “cocktail” of nucleoside reverse transcriptase inhibitors, non-nucleoside HIV reverse transcriptase inhibitors, and protease inhibitors. For example, a pharmaceutical composition including the replication defective recombinant HIV-1 particles and an HDAC inhibitor may be coadministered with a cocktail of two nucleoside reverse transcriptase inhibitors (e.g., ZIDOVUDINE (AZT) and LAMIVUDINE (3TC)), and one protease inhibitor (e.g., INDINAVIR (MK-639)).

Coadministration in the context of this invention is defined to mean the administration of more than one therapeutic agent in the course of a coordinated treatment to achieve an improved clinical outcome. Such coadministration may also be coextensive, that is, occurring during overlapping periods of time.

Pharmaceutical compositions described herein can be formulated by standard techniques using one or more physiologically acceptable carriers or excipients. Suitable pharmaceutical carriers are described herein and in “Remington's Pharmaceutical Sciences” by E. W. Martin. The small molecule compounds of the present invention and their physiologically acceptable salts and solvates can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets and capsules can be administered orally, rectally or vaginally.

For oral administration, a pharmaceutical composition or a medicament can take the form of, for example, a tablets or a capsule prepared by conventional means with a pharmaceutically acceptable excipient. Preferred are tablets and gelatin capsules comprising the active ingredient, i.e., a small molecule compound of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Compounds described herein can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In one embodiment, a pharmaceutical composition is administered to a subject, preferably a human, at a therapeutically effective dose to prevent, treat, or control a condition or disease as described herein, such as HIV.

The dosage of active compounds administered is dependent on the species of warm-blooded animal (mammal), the body weight, age, individual condition, surface area of the area to be treated and on the form of administration. The size of the dose also will be determined by the existence, nature, and extent of any adverse effects that accompany the administration of a particular small molecule compound in a particular subject. Typically, a dosage of the active compounds of the present invention is a dosage that is sufficient to achieve the desired effect. Optimal dosing schedules can be calculated from measurements of compound accumulation in the body of a subject. In general, dosage may be given once or more daily, weekly, or monthly. Persons of ordinary skill in the art can easily determine optimum dosages, dosing methodologies and repetition rates.

In another embodiment, a pharmaceutical composition including the replication defective recombinant HIV-1 particles is administered in a daily dose in the range from about 0.1 mg per kg of subject weight (0.1 mg/kg) to about 1 g/kg for multiple days. In another embodiment, the daily dose is a dose in the range of about 5 mg/kg to about 500 mg/kg. In yet another embodiment, the daily dose is about 10 mg/kg to about 250 mg/kg. In yet another embodiment, the daily dose is about 25 mg/kg to about 150 mg/kg. A preferred dose is about 10 mg/kg. The daily dose can be administered once per day or divided into subdoses and administered in multiple doses, e.g., twice, three times, or four times per day.

To achieve the desired therapeutic effect, compositions described herein may be administered for multiple days at the therapeutically effective daily dose. Thus, therapeutically effective administration of compounds to treat a condition or disease described herein in a subject requires periodic (e.g., daily) administration that continues for a period ranging from three days to two weeks or longer. Typically, compounds will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the compounds are not administered daily, so long as the administration is repeated frequently enough to maintain a therapeutically effective concentration of the compounds in the subject. For example, one can administer the compounds every other day, every third day, or, if higher dose ranges are employed and tolerated by the subject, once a week. A preferred dosing schedule, for example, is administering daily for a week, one week off and repeating this cycle dosing schedule for 3-4 cycles.

Optimum dosages, toxicity, and therapeutic efficacy of such compounds may vary depending on the relative potency of individual compounds and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the HIV infected cells to minimize potential damage to normal cells and, thereby, reduce side effects. In addition, combinations of compounds having synergistic effects described herein can be used to further reduce toxic side effects of one or more agents comprising a pharmaceutical composition of the invention.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of compounds is from about 1 ng/kg to 100 mg/kg for a typical subject.

Following successful treatment, it may be desirable to have the subject undergo maintenance therapy to prevent the recurrence of the condition or disease treated.

Although the forgoing invention has been described in some detail by way of illustration and example for clarity and understanding, it will be readily apparent to one ordinary skill in the art in light of the teachings of this invention that certain variations, changes, modifications and substitution of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present invention has a wide variety of applications. The invention is further illustrated by the following examples, which are only illustrative and are not intended to limit the definition and scope of the invention in any way.

### Example 1

### Yeast Based HIV-1 Cloning System

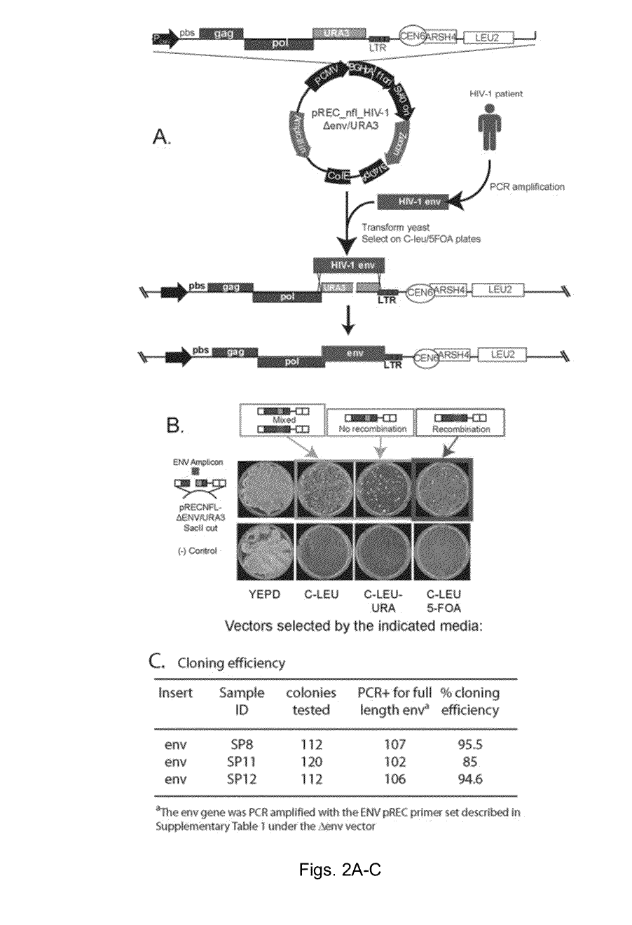

A universal HIV-1 cloning vector, pREC_nfl_HIV-1Δproteome/URA3 was constructed where the URA3 gene replaces the entire HIV-1 coding sequence. Details of this system are shown in FIG. 2A. To insert the full HIV-1 coding region, the HIV-1 genome is transformed with pREC_nfl_HIV-1Δproteome/URA3 vector into a specific S. cerevisiae strain (FIGS. 2A&B) then grown of FOA+/leu-plates. FOA is converted into the toxic anabolite unless URA3 is replaced by HIV-1 DNA genome via homologous recombination/gap repair (FIGS. 2B &C). This system has several advantages over existing technology:
(1) One step insertion of a large PCR amplicon into a vector.
(2) URA3 negative selectable system ensures that 98% of colonies contain the correct, in-frame insert
(3) Efficiency of yeast recombination is such that recombination between one PCR product and vector yields >10,000 FOA resistant colonies

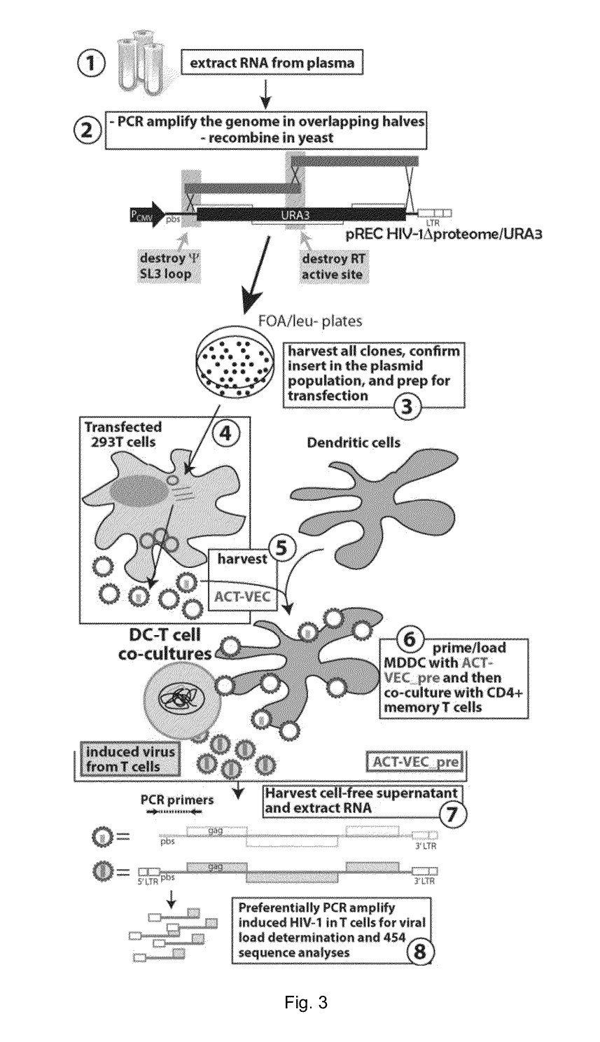

This cloning system is the technological backbone for our ACT-VEC preparation. Our pREC HIV-1 nfl plasmid upon 293T transfections produces vectors that:
(1) lack 5′LTR and as such, cannot initiate reverse transcription (FIG. 2A)
(2) lack a functional reverse transcriptase enzyme (FIG. 3)
(3) lack genomic RNA due to deletion of Ψ packaging element (FIG. 3)
(4) contains full complement of HIV-1 proteins in the correct stoichmetry
(5) are dead but morphologically identical to wildtype

### Example 2

We previously 1) utilized yeast-based cloning technology to generate a SHIV-based vaccine containing 5 to 500 HIV-1 intersubtype A/D functional env recombinants; 2) immunized mice with these SHIVenvA/DΔgagpol vaccines to determine the humoral immunogenicity and breath of possible neutralizing antibodies by screening sera for inhibitory activity against various primary HIV-1 isolates and different intersubtype envA/D recombinant viruses and screening for neutralizing monoclonal antibodies; and 3) studied the humoral and cellular immune responses in macaques vaccinated with SHIVenvA/DΔgagpol vaccines.

### Results

1) Most of gp120 recombinants are not functional.

2) The nonfunctional gp120 glycoprotein is due to the inconsistent C1/C5 and gp41.

3) Primary env-based vaccine elicits broader and more potent humoral immune response than the intersubtype env-based vaccine.

4) Inoculation of multiple SHIVenv viruses in macaques elicits broad neutralizing activity.

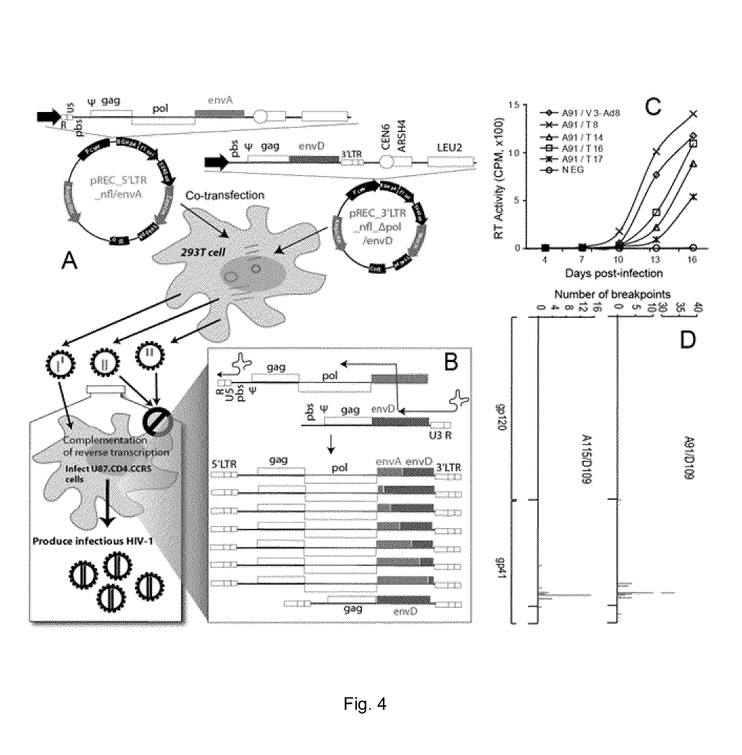

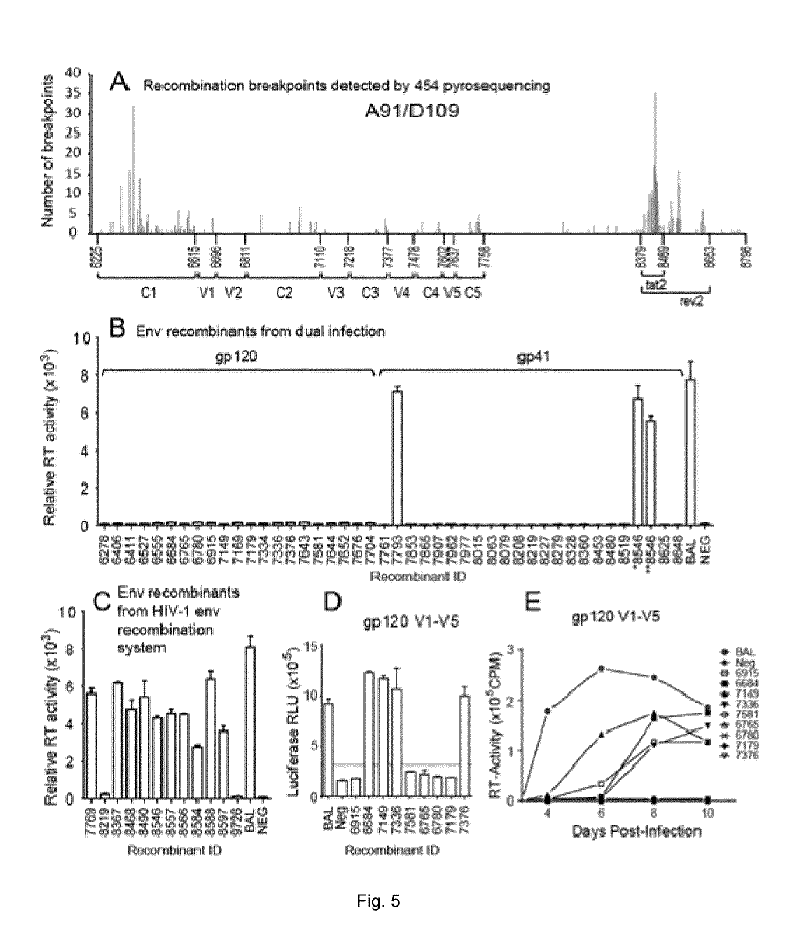

We successfully established an HIV env recombination system which can generate pure and functional intersubtype env recombinants between different HIV-1 subtypes. This system is composed of two vectors which can produce complementary HIV-1 subgenomic RNAs (sgRNA) (FIG. 4A). Upon co-transfection, the produced virus particle packaged with the two different sgRNA is able to complete the process of reverse transcription through recombination between the two sgRNAs. Only the recombination within env regions can produce the intact HIV-1 genome and the corresponding infectious virus will be screened through further propagation (FIG. 4B). This system was used to generate more than 100 HIV env clones, most of which were confirmed to be functional recombinants through fusion assays, and generation of infectious viruses (FIG. 4C). However, these env recombinants all contain breakpoints in gp41, but not in gp120 (FIG. 4D). On the other hand, the recombinants generated through the classical dual infection system contain breakpoints in both gp120 and gp41 regions (FIG. 5A), but none of the gp120 recombinants were functional in terms of eliciting membrane fusion and supporting virus replication (FIG. 5B, C). Interestingly, through systematic functional analysis of gp120/gp41 proteins, we found there is a conserved interplay between the C1 and C5 regions of gp120 and the extracellular domains of gp41, and introduction of consistent C1 and C5 regions could convert the nonfunctional env recombinants into fully functional ones (FIG. 5D, E).

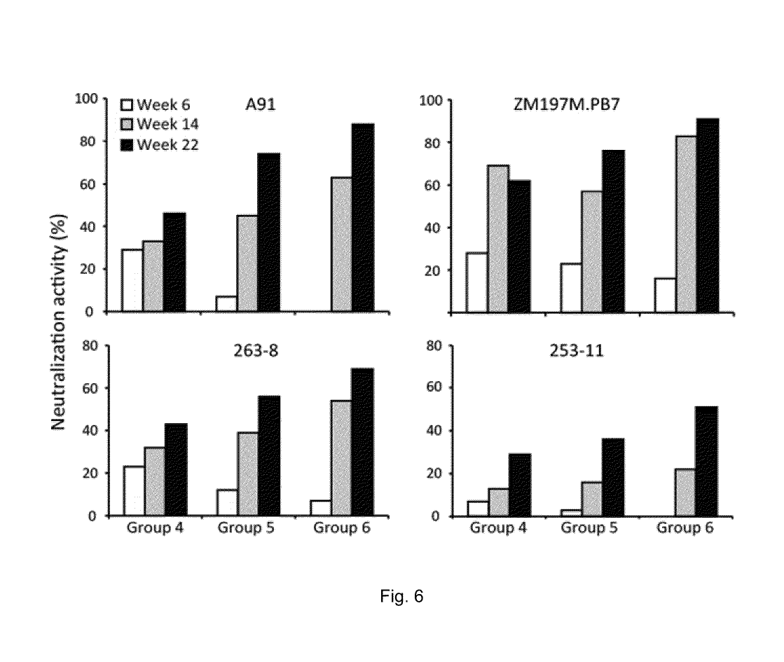

As we originally planned to utilize functional HIV-1 env intersubtype recombinants as anti-HIV vaccine candidates, in the first three years of last project, we sought to determine the possible mechanisms why most of the HIV-1 env intersubtype recombinants with breakpoints in gp120 were not functional. In the meantime, we still compared the humoral immune responses in a huCD4 B cell transgenic mouse model vaccinated with primary or recombinant env-based multivalent vaccines, and found that primary env-based multivalent vaccines with a sequential vaccination strategy elicited the broadest humoral immune responses (FIG. 6) including HIV-1 subtype A (e.g., A91), B (e.g., DJ263.8, and ZM197M.PB7), and CRF02-AG (e.g., 253-11). Please note that 253-11 is a Tier 3 isolate which is usually difficult to neutralize.

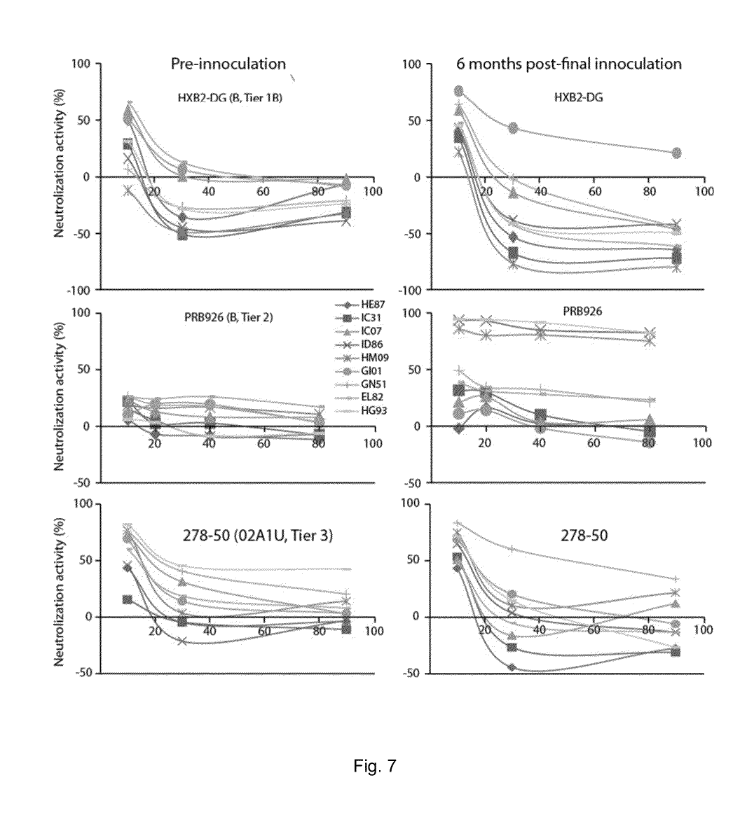

In a SHIVenv infection experiment for generation of infectious SHIVenv viruses, we also found that macaques inoculated with multiple SHIVenvB (containing 20 different subtype B envs) or SHIVenvC (containing 15 different subtype C envs) elicited humoral immune response against other subtype B (e.g., HXB2.DG) and other subtype viruses (e.g., 02A1U) (FIG. 7). The sequential vaccination with primary env-based SHIV vaccines in macaques also showed the strongest humoral immune response when compared with other vaccination strategies, including repeated vaccination with the same 80 multivalent SHIV pseudotyped viruses (FIG. 8).

The huCD4 B cell transgenic mouse model has many advantages for studying humoral immune responses elicited by multivalent vaccines. This special animal model has B cells that express human CD4 molecules on the cell surface which can bind to HIV gp120 glycoproteins, inducing proper conformational changes which expose the hidden viral epitopes, thus possibly resulting in more effective immune response than other mouse or animal models. However, due their small size, mice, cannot provide enough serum (˜200 ul/mouse) for investigating the breadth of neutralization activity. Therefore, we combined the mouse and rabbit models to better understand the breadth of the immune response induced by multivalent anti-HIV vaccines. The rabbit model will not only provide larger amount of sera for a variety of immune study, but also has other advantages, such as less background reactivity to testing antigens, and they are highly immunogenic in response to various immunizations, and produce high titer antibody responses. It was shown that only RMAbs were able to provide high-quality detection using certain difficult epitopes, such as those in tissue section samples and HIV particles.

### Generation of Various HIV/SHIV Pseudotyped Viruses for Vaccination of Mice and Rehsus Macaques

By using our HIV-1 inter-subtype recombination system, so far, we have totally generated ˜600 A/D envelope recombinants, and confirmed >400 clones by sequence analyses. Interestingly, we found that the breakpoints of env recombinants from dual infection system spread across the entire envelope sequence, but the ones from the recombination system are mainly confined within gp41 region. For future screening of the broadly anti-HIV-1 neutralizing antibodies, we have generated more than 100 HIV-1 chimeric viruses containing HIV-1 A/D envelope recombinants. Most of the env recombinants from the recombination system are functional, and yielded infectious HIV-1 env chimeric viruses. However, most of the env recombinants from dual infection system are not functional, and yield non-infectious chimeric viruses. Interestingly, we found that, when we cloned only C2-V4 sequences of gp120 recombinants into NL4-3 backbone, the chimeric envelopes became functional and produced infectious virus particles. We suspect that there is an interaction between C1 and C5 region which might be necessary in maintaining the envelope conformation and play an important role in viral entry.

### Generation of Various SHIV Pseudotyped Viruses for Vaccination of Rehsus Macaques

By using the vaccine system, we've cloned 100 A/D envelope recombinants, as well as 100 primary envelope sequences (subtype A, B, C and D) into pREC_SHIV_{KB9}_{_{—}}Δgagpol_Δenv/URA3 to generate pREC_SHIV_{KB9}_{_{—}}envA/D (or env A, B, C, D)_Δgagpol vector for production of multivalent anti-SHIV vaccines for macaque experiment. The same system has also been developed for HIV-1 vaccine vectors, i.e., pREC_HIV_gag/pol, pREC_HIV_Δgagpol, and pCMV_HIV_cpltRU5, and the testing has been successfully accomplished.

### Construction of HIV Vaccine Vector and Mouse Immunization Experiment

We successfully generated >100 various pREC_nfl_HIVenvX vectors (which only lacks of 5′LTR sequence) and produced the corresponding virus-like particles for mouse immunization experiment. We have started utilizing these multivalent non-infectious HIV-1 virus particles containing either parental envs or recombinant envs to vaccinate two different mouse models, BLAB/C wild-type and huCD4 B cell transgenic mice, for investigation of the breadth of immune response and screening of broadly reactive anti-HIV neutralizing antibodies. To do this, we used increasing number of env immunogens (1, 5, and 25) to immunize wild-typed BLAB/C mice or huCD4+ B cell transgenic mice. We also applied a strategy “sequential immunization”, i.e., vaccinating mice with total 25 immunogens, but 5 for each time for 5 times, which might be able to accelerate the maturation of B cell response.

The preliminary data showed that the multivalent anti-HIV vaccine can elicit higher titer of anti-gp160 antibody response in huCD4 B cell transgenic mice in 6 weeks post vaccination with either one or five env recombinants-based vaccines. We then utilized one HIV-1 subtype A strain (i.e., A91) and three env clones (i.e., ZM197M.PB7, 263-8, and 253-11) from the standardized tiers of HIV-1 strains for detection of anti-HIV NAbs to the neutralization activity of the immune sera from various groups of immunized mice. The sequential immunization strategy can elicit gradually increased humoral immune response, and the huCD4 B cell transgenic mice sequentially immunized with 25 parental env-based vaccines produced broadest humoral immune response.

Generation of Hybridoma Cells with Anti-HIV Vaccine Immunized Mice

By using the established hybridoma technology, we have successfully established the method of generating hybridoma cells, and generated over 10 anti-gp41-producing hybridoma cell clones from the gp41 immunized mice. Based on our sequencing data, so far, we have screened three hybridomas secreting distinguishable IgG antibodies with specificity. We utilized the mice from WT mouse vaccinated with 25 env recombinants-based vaccines through sequential immunization strategy and huCD4 transgenic mouse vaccinated with 25 parental envs-based vaccines through sequential immunization strategy, and generated over 100 hybridomas producing anti-gp160 antibodies.

Macaque Experiment for Investigation of Immune Response Elicited with Multiple SHIV Viruses

We started the macaque experiment to investigate the immune responses elicited with multiple SHIVgp120B or SHIVgp120C viruses, as well as the possible virus replication. Briefly, nine male monkeys are divided into 3 groups. Group 1 was infected with 500 TCID50 of SHIVKB9_gp120B3 (confirmed infectivity in the previous expt) through intravenous route, group 2 with 500 TCID50 of a pool of 20 different SHIVKB9_gp120B, and group 3 with 500 TCID50 of a pool of 14 different SHIVKB9_gp120C. The detection of viral load showed that, after the initial three inoculations, none of these nine macaques were productively infected, possibly due to the low inoculation of SHIVgp120 viruses (i.e., 500 TCID50). Thus, we used 100 times more of the same SHIVgp120 viruses to inoculate these macaques one more time. Interestingly, we found that the Group 1 macaques, which were inoculated with SHIVgp120B3 only, were all productively infected, but the macaques in other three groups (including a new recruited group) injected with pools of SHIVgp120Bs or SHIVgp120Cs, were not infected. We suspect that there might be an interference among different HIV strains which decreases the virus entry efficiency, so we are now giving a single SHIVgp120 virus to the individual macaques, which might result in efficient infection. We are also planning to give another two high dose of SHIVgp120 inoculations (either single B3 or pool) to macaques in Group 1, 2, and 3, and then challenge them with SHIVgp120B3 to investigate the difference of protection from virus infection between a single and multiple virus inoculations.

### Macaque Immunization Experiments

We started the macaque immunization experiments. 24 macaques are divided into 4 groups that are immunized with various env-based pseudovirus vaccines, i.e. Group 1 receives 1 HIV-1 env-based vaccines (i.e., envB3), Group 2 receives 4 different subtype env-based vaccines (i.e., one of each subtype A, B, C, and D), group 3 receives 80 different envs (i.e., 20 of each subtype A, B, C, and D), and group 4 receives sequential immunization, i.e., each time receives 20 different subtype env-based vaccines. All of the groups will be immunized 5 times, including a final boost, through LN route and with R848 as an adjuvant. Another 6 macaques are used a control group and receives a control solution and R848 for 5 times. After the final immunization, the macaques will be challenged with a number of SHIV viruses to investigation the protection efficiency provided by the different vaccines and vaccination strategies. The blood samples will be collected to detect the humoral and cellular immune response, as well as the viral load. The peripheral lymph node and intestinal biopsies will also be performed.

From the above description of the invention, those skilled in the art will perceive improvements, changes and modifications. Such improvements, changes, and modifications are within the skill of the art and are intended to be covered by the appended claims.

Having described the invention, the following is claimed:

1. A method for inducing an immune response against HIV in a subject, the method comprising the steps:
preparing first and second HIV-1 protein coding sequences;
introducing the first and second HIV-1 protein coding sequence into first and second expression constructs using yeast homologous recombination;
transfecting a cell with the first and second, wherein the HIV-1 particle is secreted by the cell; and
administering the secreted HIV-1 particle and a pharmaceutically acceptable carrier to the subject, wherein the secreted HIV-1 particle stimulates an immune response.

2. The method of claim 1, the method further including obtaining a biological sample from the subject, wherein the biological sample includes a blood plasma sample that includes HIV-1 RNA and wherein at least one of the first and second HIV protein coding sequences is prepared from the HIV-1 RNA.

3. The method of claim 1, wherein the HIV-1 particle secreted by the cell is a defective HIV-1 particle including env, gag and pol proteins in the correct stoichiometry and is morphologically indistinguishable from a wild type HIV-1.

4. The method of claim 1, further comprising the step of harvesting the HIV-1 particle.

5. The method of claim 2, wherein the preparation of the at least on the first and second HIV-1 protein coding sequence from a sample obtained from the subject includes reverse transcribing the HIV-1 RNA to produce HIV-1 cDNA and amplifying a fragment of the HIV-1 cDNA, the amplified fragments corresponding to a portions of an HIV-1 protein coding RNA sequence.

6. The method of claim 1, wherein the step of introducing the at least one HIV-1 protein coding sequence into at least one expression construct using yeast homologous recombination comprises providing a plasmid expression vector including a near-full length HIV-1 genome having a yeast uracil biosynthesis gene (URA3) in place of a gp120/gp41 HIV-1 envelope protein coding sequence and replacing the yeast uracil biosynthesis gene with an HIV-1 envelope protein coding sequence prepared from the subject sample.

7. The method of claim 6, the HIV-1 envelope protein coding sequence encoding HIV gp120 and an N-terminal portion of gp41.

8. The method of claim 6 wherein the HIV-1 envelope protein coding sequence does not encode a functional portion of the cytoplasmic domain of gp41.

9. The method of claim 1, wherein the step of introducing the at least one of the first and second HIV-1 protein coding sequence into at least one of the first and second expression constructs using yeast homologous recombination comprises providing a plasmid expression vector including a near-full length HIV-1 genome having a yeast uracil biosynthesis gene (URA3) in place of a HIV-1 gag/pol protein coding sequence and replacing the yeast uracil biosynthesis gene with an HIV-1 gag/pol protein coding sequence prepared from the subject sample.

10. The method of claim 1, the at least one expression construct comprising a promoter operably linked to the HIV-1 protein coding sequence.
